(19) United States
(12) Patent Application Publication
(10) Pub. No.: US 2004/0034006 A1
(43) Pub. Date: Feb. 19, 2004

(54) Method of inactivating viral particles in a blood product

(76) Inventors: Thomas Marinus Albert Remko Dubbelman, Alphen a/d Rijn (NL); Johannes Wilhelmus Maris Lagerberg, Leiden (NL)

Correspondence Address: PEACOCK MYERS AND ADAMS P C, P O BOX 26927, ALBUQUERQUE, NM, 871256927

(21) Appl. No.: 10/398,925

(22) PCT Filed: Oct. 15, 2001
(86) PCT No.: PCT/NL01/00758

(30) Foreign Application Priority Data
Oct. 13, 2000 (EP) 00203559.0

(51) Int. Cl. A61K 31/555; A61K 31/409

(52) U.S. Cl. 514/185; 514/410

(57) ABSTRACT

A method is provided for inactivating viral and/or bacterial contamination in blood cellular matter, such as erythrocytes and platelets, or protein fractions. The cells or protein fractions are mixed with chemical sensitizers and irradiated with, for example, UV, visible, gamma or X-ray radiation.

The present invention relat s to a method of inactivating viral particles in a blood product comprising the steps of

combining said blood product with a photosensitiser, and

activating the photosensitiser.

The transmission of viruses (HIV, hepatitis B and C) by transfusion with infected blood products presents a serious health problem.

U.S. Pat. No. 5,360,734 describes a method of inactivating viral pathogens in a body fluid, such as plasma, red cells, platelets, leukocytes and bone marrow.

The method comprises adding the photosensitiser to the blood product and irradiation with light to activate the photosensitiser, thereby inactivating viral pathogens. The method disclosed in U.S. Pat. No. 5,360,734 is in particular aimed at reducing the influence of plasma proteins in order to increase the stability of the red cells.

The object of the present invention is to provide a further method of improving the stability of a blood product and in particular red cells and platelets, which method may or may not be used in conjunction with the improvement disclosed in U.S. Pat. No. 5,360,734.

Thus, the present invention relates to a method according to the preamble characterized in that as the photosensitiser a compound is used with the formula I

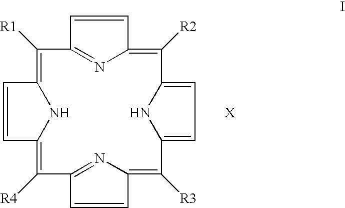

wherein R₁ is chosen from the group consisting of

hydrogen,

(C₁-C₂₀)alkyl, (C₁-C₂₀)alkoxy, (C₁-C₂₀)acyl, (C₁-C₂₀)acyloxy, (C₂-C₂₀)alkenyl, or (C₂-C₂₀)alkynyl, each of which may be linear or branched and each of which may be substituted with one or more groups chosen from

hydroxyl,

amino which may be substituted with 1 to 3 groups chosen from (C₁-C₂₀)alkyl, (C₂-C₂₀)alkenyl, (C₁-C₂₀)alkoxy, (C₂-C₂₀)alkynyl, and —(R₅-Z)ₘ-R₆ where R₅ is (CH₂)ₙ, Z is O or S, and R₆ is (C₁-C₂₀)alkyl and m and n are, independently, 1-10, each substituent group of the amino group may be linear or branched and each of these may be substituted with one or more groups chosen from hydroxyl, and a halogen atom,

nitril, and

a halogen atom,

(C₆-C₂₀)aryl, and (C₆-C₂₀)heterocyclic aryl group each of which may be substituted with one or more groups chosen from

hydroxyl,

amino which may be substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom,

nitril,

a halogen atom, and

(C₁-C₁₀)alkyl, (C₁-C₁₀)alkoxy, (C₂-C₁₀)alkenyl,

R₂, R₃ and R₄ representing a pyridinium group, the nitrogen of which is substituted with a (C₁-C₄)alkyl group, and wherein X is a pharmaceutically acceptable counterion.

Surprisingly, it has been found that by using a photosensitising compound as disclosed above, the damage to the blood product is strongly reduced. This is evident from experiments which are generally recognized as models to determine the extend of damage to a blood product. In particular this was shown in experiments where the hemolysis and functional parameters are measured. In general, if R₁ is an aliphatic group, short chains will be preferred, such as those having 1-6 atoms.

In the present invention, the term “blood product” is to be understood as any solution comprising at least one of i) red blood cells and ii) platelets. Other cell types may be present and the solution, while being an aqueous solution, may contain proteinaceous components, salts, stabilizers, anti-coagulation agents such as citric acid or heparin, as is generally recognized in the art.

The term “photosensitizer” is, as well recognized in the art, a substance which absorbs light energy as a result of which the photosensitizer is activated. The activated photosensitizer can subsequently react with other compounds. This may result in the photosensitizer being modified or inactivated, but more likely the photosensitizer will return to its original state (before it was activated with light), so as to form a photocatalytic cycle in which the photosensitizer can be used again. In effect, the light energy absorbed is used for the inactivation of viral particles.

The term “viral particle” is understood to mean any RNA or DNA virus, single- or double-stranded and with a membrane or proteinaceous coat that may occur in a blood product. Examples are HIV and hepatitis B.

The term “pharmaceutically acceptable counterion” is understood to be any inorganic or organic negatively charged counterion such as OH⁻, Cl⁻, acetate or citrate. It goes without saying that there are as many counterions X as needed to neutralise the positive charge of the actual active compound I.

Ben-Hur E. et al disclose in Transfusion 3S(5), pp401-6 (1999) a method of inactivating virus present in a solution comprising red blood cells. To protect the red blood cells, a protective agent has to be added. As a result of this, which applicants have not been able to replicate due to the difficulties of dissolving the protecting agent Trolox, hemolysis was over 1% after 10 days of storage. In contrast, the method according to the present inv ntion results in less than 1% hemolysis above the control after 5 weeks in the absence of a protecting agent. Please note that the xperiments presented below were conducted in glass test tubes, instead of special containers for blood designed to reduce damage to blood cells. Of course, the method according to the present invention still allows for the addition of protective agents, such as disclosed in PCT/NL99/00387.

According to a first embodiment R₁ is a (C₆-C₂₀)aryl group substituted with an amino which may be substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom. Preferably the aryl group is a trialkyl aminophenyl group where alkyl is independently (C₁-C₃)alkyl.

It has been found that quaternary nitrogen atoms in groups as defined above are very suitable for eliminating viral particles, gram positive and gram negative bacteria, while maintaining the integrity of the blood product.

The invention will now be elucidated with reference to the exemplary embodiments and the drawing, in which

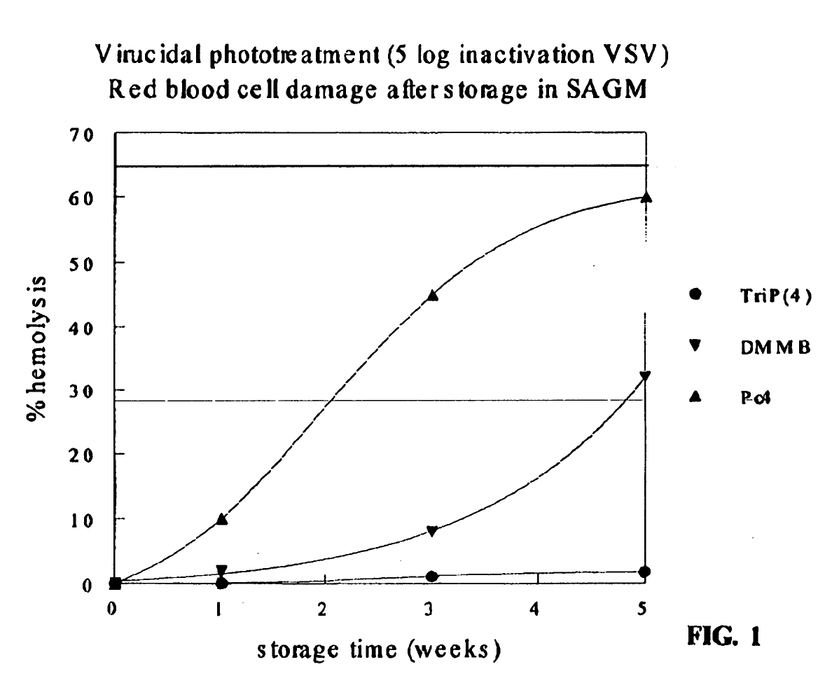

FIG. 1 shows the degree of hemolysis using a compound in accordance with the present invention (TriP4), and two control compounds;

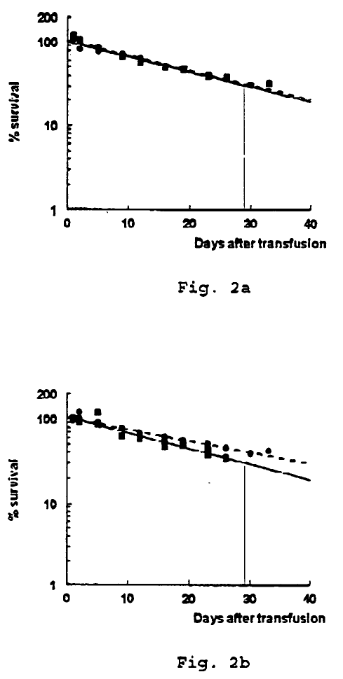

FIGS. 2a and b show RBC survival after transfusion immediately after treatment of RBCC prepared from freshly withdrawn blood of two Rhesus monkeys. ▪, Control; O, treated;

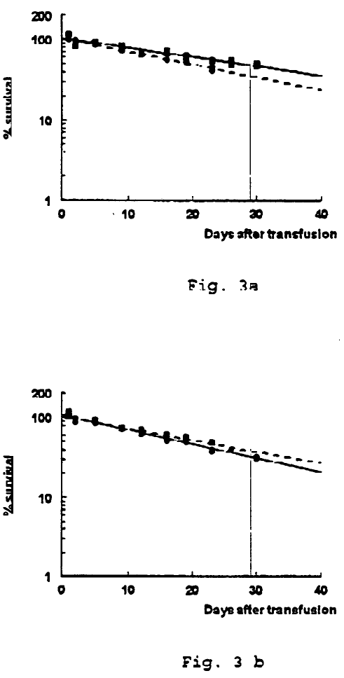

FIG. 3 show RBC survival after transfusion after 5 weeks of storage after treatment of RBCC prepared from freshly withdrawn blood of two monkeys of FIG. 2. ▪, Control; O, treated;

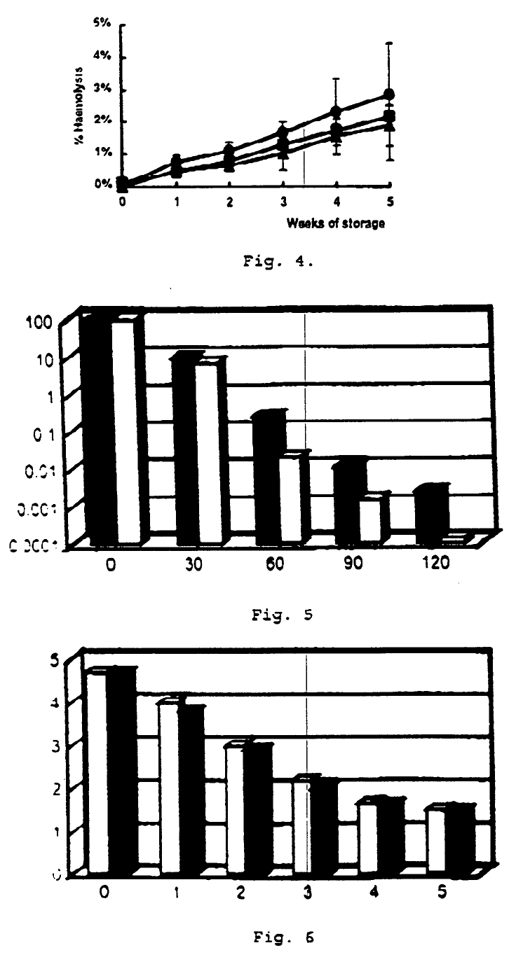

FIG. 4 shows haemolysis during dark storage after photodynamic treatment of RBCC prepared from freshly withdrawn blood from the two monkeys of FIG. 2. Photodynamic treatment was performed immediately after withdrawal. ▪, Control; Δ, dark control; O, treated;

FIG. 5 shows inactivation of two types of bacteria (black, Gram-positive; while, Gram-negative) with varying amounts of light; and

FIG. 6 shows the ATP content of treated (black) and untreated (white, control) red blood cells.

### EXAMPLES

### Blood Product

The blood product used throughout these studies were standard blood cell concentrates (RBCC) without buffy coat, and were provided by the Sanquin Blood Bank, Leiden-Haaglanden, Leiden, the Netherlands, and obtained from healthy human volunteers. Blood from these donors (500±50 ml) was collected in 73 ml citrate-phosphate-dextrose (CPD) in quadruple polyvinylchloride bags (NPBI, Emmer-Compascuum, the Netherlands). RBCCs were prepared as described before (Novotny, 1992). The hematocrit was adjusted to 56±4 with the preservative saline-adenine-glucose-mannitol (SAG-M). SAG-M contains 150 mM NaCl, 50 mM glucose, 29 mM mannitol and 1.25 mM adenine. The number of white cells was 1.8±0.9*10⁹ cells/l, as determined on an automatic cell counter (Sysmex K1000, TOA Medical Electronics, Kobe, Japan).

### Photodynamic Treatment of Virus Containing RBCC

Stock solutions of vesicular stomatitis virus (VSV) (San Juan strain, kindly provided by the Department Virology, Leiden University Medical Center) were added to RBCC such that the volume of the spike was <10% of the total volume of RBCC, and the number of virus particles was about 10⁵/ml. Sensitizers were added to a final concentration of 5 μM aluminium phthalocyanine tetrasulfonate (AIPcS₄, Phorphyrin Products, Logan, Utah, USA), 0.45 μM silicon phthalocyanine, HO-SiPc(CH₃)₂(CH₂)₃N(CH₃)₂, (Pc4, donated by Dr. M. E. Kenney, Case Western Reserve University, Cleveland Ohio, USA), 15 μM dimethylmethylene blue (DMMB, ICN Biomedicals BV, Zoetermeer, the Netherlands), and, in accordance with the invention, 25 μM monophenyl-tri(N-methyl-4pyridyl)porphyrin chloride (TriP(4), Mid-Century, Posen, Ill., USA). The suspensions were thoroughly mixed and divided into 6-ml aliquots in polystyrene culture dishes with a diameter of 9 cm (Greiner, Alphen a/d Rijn, the Netherlands), and agitated at room temperature on a horizontal reciprocal shaker (60 cycles/min, GFL, Burgwedel, Germany) for 5 min. in the dark. The dishes (one dish per time point) were illuminated from above with a 300 W halogen lamp (Philips, Eindhoven, the Netherlands). The light passed through a 1-cm water filter, to avoid heating of the samples. A cut-off filter, only transmitting light with wavelengths above 600 nm, was used in all experiments. The irradiance at the cell layer was 35 mW/cm², as measured with an IL1400A photometer equipped with a SEL033 detector (International Light, Newburyport, Mass., USA). After treatment the cells were suspended in AS3 (70 mM NaCl, 61.1 mM glucose, 2.22 mM adenine, 2 mM citric acid, 20 mM Na-citrate, and 15.5 mM Na-phosphate, pH 5.8) and stored for a period of up to 5 weeks. The damage to the red blood cells was established by measuring the degree of hemolysis over this period. To compare the results with those of Ben-Hur (supra), the solution was illuminated to achieve a 100,000 fold decrease in viral load. As is shown in FIG. 1, the method according to the present invention achieves a better result after 10 days than the method of Ben-Hur, even in the absence of a protecting agent.

Table 1 shows similar experimental results for photodynamic inactivation of vesicular stomatitis virus (VSV) spiked in a 56% red blood cell concentrate (RBCC) in SAG-M. The concentration of the various photosensitisers was 25 μM. The intensity of the light source was 35 mW/cm².

TABLE 1

| | infectious particles (% of control) | | | | | | |
|---|---|---|---|---|---|---|---|
| light dose | Sylsens | | Sylsens | Sylsens | Sylsens | Sylsens | Sylsens |
| (kJ/m²) | A | Sylsens | C | D | E | O | R |
| 0 | 100 | 100 | 100 | 100 | 100 | 100 | 100 |
| 60 | 10 | 0.126 | 0.79 | 3 | | 0.398 | 1.58 |
| 90 | | 0.001259 | | | | | |
| 120 | 0.25 | | 0.015 | 0.08 | | 0.001 | 0.079 |
| 150 | | | | 0.013 | | | |
| 180 | 0.01 | | | | | | 0.001 |
| 240 | 0.00158 | | | | 0.08 | | |
| 300 | | | | | 0.01 | | |
| 360 | | | | | 0.0025 | | |

Table 2 shows the effect on haemolysis of red cells upon storage at 4° C., when a 56% RBCC in SAG-M is photodynamically treated with 25 μM of the various photosensitisers and a light dose needed to inactivate 5 logs of VSV. The intensity of the light source was 35 mW/cm².

TABLE 2

| | % haemolysis | | | | | |
|---|---|---|---|---|---|---|
| storage time | | Sylsens | | Sylsens | Sylsens | Sylsens |
| (days) | Control | A | Sylsens | C | D | O |
| 0 | 0 | 0 | 0 | 0 | 0 | 0 |
| 7 | 0.36 | 0.87 | 0.57 | 31.5 | 50 | 0.61 |
| 21 | 1.06 | 3.9 | 2.85 | 72 | 77 | 4.5 |
| 35 | 2.26 | 15.4 | 7.07 | 66 | 85 | 17 |

These representative compounds show that compounds with fewer charged groups (such as 2) cause more damage to the erythrocytes, whereas the representative compound with 3 charges performs best, both with respect to haemolysis and infectious particle inactivation.

List of photosensitisers:

| | |
|---|---|
| Sylsens A | meso-tetra-(N-methyl-4-pyridyl)-porphyrin chloride |
| Sylsens | mono-phenyl-tri-(N-methyl-4-pyridyl)-porphyrin |
| | chloride |
| Sylsens C | trans-diphenyl-di-(N-methyl-4-pyridyl)- |
| | porphyrin chloride |
| Sylsens D | cis-diphenyl-di-(N-methyl-4-pyridyl)-porphyrin |
| | chloride |
| Sylsens E | triphenyl-mono-(N-methyl-4-pyridyl)-porphyrin |
| | chloride |
| Sylsens R | tetra-(N-2-hydroxy-ethyl-4-pyridyl)-porphyrin |
| | chloride |
| Sylsens O | tetra(4-N, N, N-trimethylanilinium)-porphyrin |
| | chloride |

An experiment was performed to test the effect of photosterilisation of red blood cell concentrates for in vivo purposes. To this end the in vivo survival of RBC photodynamically treated with SYLSENS was studied. Red blood cells obtained from 2 Rhesus monkeys (Macaca mulata) were labeled with ⁵¹Cr according to standard procedures. Each monkey received its own blood, treated or not with Sylsens in accordance with the procedure desribed above. If the cells were stored, the labeling with ⁵¹Cr took place just before the cells were re-infused. The results are shown in table 3, with recovery (%) of red blood cells 24 h after re-infusion, either re-infused immediately after treatment (I), or after 5 weeks of storage after treatment (II).

| Monkey | Control I | Treated I | Control II | Treated II |
|---|---|---|---|---|
| BB26 | 73 | 75 | 81 | 71 |
| CO33 | 88 | 102 | 86 | 72 |

The results in table 3 show that the erythrocytes treated according to the present invention are for practical purposes entirely suitable for in vivo use. FIGS. 2 and 3 show the data of Table 3 (as well as intermediate and subsequent data) for each of the monkeys (Figs. a for monkey BB26, and b for monkey CO33). There is no difference in survival between treated (O) and non-treated (▪) cells.

FIG. 4 shows in vitro haemolysis data, obtained during dark storage after photodynamic treatment of RBC prepared from freshly withdrawn blood from monkeys CO33 and BB26. Photodynamic treatment was performed immediately after withdrawal. It is clear that the degree of lysis is very satisfactory, and it would well meet the requirements if the treatment is not performed in test tubes but special blood containers designed to limit damage to blood cells. Experiments with human red blood cells gave identical results.

FIG. 5 shows the depencency on the amount of light (in kJ/m²) on the inactivation of P. aeruginosa and S. aureus in the presence of 25 uM Sylsens. The percentage drops from 100% to about 0.001 or less.

FIG. 6 shows the ATP content in AS3 (umol/g haemoglobin determined with standard procedures of the Central Clinical & Chemical Laboratory of the Leiden University Medical Center) during storage (in weeks) of control () red blood cells (RBCC) and RBCC treated photodynamically with 25 uM Sylsens. Similarly, there was no difference during storage of glucose consumption, lactate production, 2,3-DPG content and pH between the control and treated RBCC (data not shown). White blood cells have found to be highly effected by the present photodynamic treatment. In particular their response to allogenic stimulation is strongly reduced, and also their capability to present antigens is adversely affected.

1. Method of inactivating viral particles present in a blood product comprising the steps of
combining said blood product with a photosensitiser, and
activating the photosensitiser,
characterized, in that as the photosensitiser a compound is used with the formula I 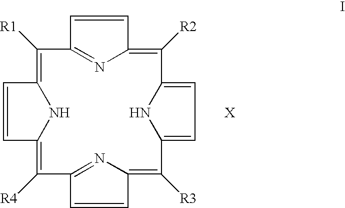
wherein R₁ is chosen from the group consisting of
hydrogen,
(C₁-C₂₀)alkyl, (C₁-C₂₀)alkoxy, (C₁-C₂₀)acyl, (C₁-C₂₀)acyloxy, (C₂-C₂₀)alkenyl, or (C₂-C₂₀)alkynyl, each of which may be linear or branched and each of which may be substituted with one or more groups chosen from
hydroxyl,
amino which may be substituted with 1 to 3 groups chosen from (C₁-C₂₀)alkyl, (C₂-C₂₀)alkenyl, (C₁-C₂₀)alkoxy, (C₂-C₂₀)alkynyl, and —(R₅-Z)ₘ-R₆ where R₅ is (CH₂)ₙ, Z is O or S, and R₆ is (C₁-C₂₀)alkyl and m and n are, independently, 1-10, each substituent group of the amino group may be linear or branched and each of these may be substituted with one or more groups chosen from hydroxyl, and a halogen atom,
nitril, and
a halogen atom,
(C₆-C₂₀)aryl, and (C₆-C₂₀)heterocyclic aryl group each of which may be substituted with one or more groups chosen from
hydroxyl,
amino which may be substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom,
a nitril,
a halogen atom, and
(C₁-C₁₀)alkyl, (C₁-C₁₀)alkoxy, (C₂-C₁₀)alkenyl, R₂, R₃ and R₄ representing a pyridinium group, the nitrogen of which is substituted with a (C₁-C₄)alkyl group, and wherein X is a pharmaceutically acceptable counterion.

2. Method according to claim 1, characterized, in that R₁ is a (C₆-C₂₀)aryl group substituted with an amino which may be substituted with 1 to 3 groups chosen from (C₁-C₂₀)alkyl, (C₂-C₂₀)alkenyl, (C₁-C₂₀)alkoxy, and (C₂-C₂₀)alkynyl, each of which may be linear or branched and each of which may be substituted with one or more groups chosen from hydroxyl, and a halogen atom.

3. Method according to claim 2, characterized in that the aryl group is a trialkyl aminophenyl group where alkyl is independently (C₁-C₃)alkyl.

4. Method according to claim 1, characterized in that the photosensitiser is monophenyl-tri(N-methyl-4-pyridyl)porphyrin chloride.
